(19) United States
(12) Patent Application Publication
(10) Pub. No.: US 2005/0266512 A1
(43) Pub. Date: Dec. 1, 2005

(54) Detection of proteases and screening for protease inhibitors

(76) Inventor: J Thomas Buckley, Victoria (CA)

Correspondence Address: KLARQUIST SPARKMAN, LLP, 121 SW SALMON STREET, SUITE 1600, PORTLAND, OR, 97204, US

(21) Appl. No.: 10/416,047

(22) PCT Filed: Nov. 7, 2001
(86) PCT No.: PCT/CA01/01561

§ 371 Date: May 5, 2003

Related U.S. Application Data
(60) Provisional application No. 60/247,160, filed on Nov. 7, 2000.

(51) Int. Cl. C12Q 1/37

(52) U.S. Cl. 435/23

(57) ABSTRACT

The present disclosure provides a method for detecting a protease, which is simple, sensitive, and capable of high throughput. The method detects a protease in a sample by measuring lysis of a liposome due to activation of a modified or inactive channel-forming agent. Also disclosed is a method for screening a test compound, to determine if the test compound can function as protease inhibitor. A method for identifying a protease cleavage site is also disclosed.

### FIELD

Herein disclosed is a method for detecting a protease in a sample. The assay detects lysis of liposomes in response to activation of a channel-forming agent. This assay is simple, sensitive, and capable of high throughput for screening.

### BACKGROUND

Proteases play an important role in the regulation of biological processes in almost every life form from virus to bacteria and to mammals. They perform critical functions in, for example, digestion, blood clotting, fertilization, viral maturation, activation of zymogen, and formation and release of hormones and growth factors. To date, more than 140 different types of proteases have been identified in mammalian cells and many other types have been found in viruses, bacteria, fungi, insects and plants.

In addition to their roles. in the regulation of cell functions, the activities of some proteases can be altered by disease processes that involve tissue injury, necrosis, inflammation, repair, degeneration or infection. Abnormally high activities of certain specific proteases are found at the sites of physical or chemical trauma, blood clots, malignant tumors, rheumatoid arthritis, inflammatory bowel diseases, gingival disease, glomerulonephritis, prostate cancer and acute pancreatitis. A number of specific proteases have been implicated as causes or contributors to Alzheimer's disease, cystic fibrosis, pulmonary emphysema, atherosclerosis, hypertension, and muscular dystrophy.

Various pathogenic viruses, bacteria, and fungi also rely on specific proteases for infection, replication, and maturation. For example, the-aspartyl protease of the human immunodeficiency virus (also known as HIV-1 protease) is translated as part of the viral Gag-Pol polypeptide and is responsible for its own processing and for releasing structural proteins and enzymes during viral replication and maturation in host cells. The serine protease NS3 of the hepatitis C virus (HCV) is responsible for the release of nonstructural proteins from the HCV polyproteins that are essential for the replication of the virus in the host cells. In addition, the viral-specific proteases (EP Application No. 514,830; Liu and Roizman, 1991) of the herpes virus (HSV) and a related protease known as assemblin (Welch et al., 1991) of the cytomegalovirus (CMV) are known for their critical role in viral replication in the host cells.

The ability to detect viral-specific, cellular-specific, or disease-specific protease activity in a simple and sensitive assay is important for biochemical characterization of these enzymes, diagnosing infectious diseases, and screening and/or identification of protease inhibitors. Conventionally, protease assays are performed with colormetric methods using common proteins such as casein or their fluorogenic derivatives as substrates. Although these assays are quick and simple, they lack the specificity to a particular protease and can only be used to estimate the proteolytic activities in crude biological samples.

Several specific protease assay methods have been developed by using cleavage-site specific substrates. For example, Sharma (U.S. Pat. No. 5,171,662) disclosed a peptide substrate that contains a specific amino acid sequence capable of being cleaved by HIV protease and an immunoassay method for detecting the cleavage product. In addition to immunoassay, other methods for detecting specific cleavage products include HPLC, electrophoresis and spectrophotometric analysis. Although the specific protease assays are sensitive, they are often cumbersome to perform and require specialized instruments.

Several cell-based systems have been developed for specific protease assays. For example, Hirowatari et al. (1995) describe a cell-based assay used to detect the activity of HCV NS3 protease using a fusion protein consisting of the NS3 cleavage site and Tax 1 protein. The fusion protein gene and a reporter gene encoding chloramphenicol transferase (CAT) were cotransfected into COS cells. The fusion protein substrate was expressed as a membrane-bound protein. Upon cleavage by NS3 protease, the release of Tax 1 activates CAT and the protease activity determined by measuring CAT activity in cell lysate. Similar cell-based, specific protease assay systems involving activation of reporter gene constructs and expression are also described by Germann et al. (U.S. Pat. No. 6,117,639), Smith et al. (1991), Dasmahapatra et al. (1992), and Murray et al. (1993). However, these cell-based protease assay systems, are limited because they require construction of complex reporter gene expression systems.

The specific protease assays described above are either cumbersome to perform, or require the synthesis of costly peptides, complex gene expression systems, and specialized instruments. None of these assays allow high throughput screening of specific proteases in biological samples and evaluation of protease inhibitors in animal models. Thus, there is a need for a simple and sensitive assay that is capable of both detecting specific proteases and high throughput screening.

### SUMMARY

The present disclosure provides a method for detecting a protease, which is simple, sensitive, and capable of high throughput. The method can detect a protease in a sample by measuring lysis of a liposome due to activation of a modified or inactive channel-forming agent. The modified channel-forming agent comprises a protease cleavage site specific for the protease to be detected. Proteolytic cleavage at the specific cleavage site by the protease in the sample results in activation of the channel-forming agent. The activated channel-forming agent, when in contact with a liposome, such as a cell, forms transmembrane pores or channels in the liposome membrane, resulting in cell lysis that can be measured. The level of cell lysis in a sample is indicative of the amount of protease present in the sample.

Also disclosed herein is a method for screening a test compound, to determine if the test compound can function as protease inhibitor. The method includes contacting an inactive channel-forming agent including a protease cleavage site specific for a protease inhibited by the protease inhibitor, with the test compound, protease, and a liposome, then measuring lysis of the liposome. The amount of lysis can be compared to a parallel sample which does not contain the test compound. If the test compound does not function as a protease inhibitor, the liposome will be lysed by the protease. If the test compound functions as a protease inhibitor, the liposome will not be lysed by the protease.

A method for identifying a protease cleavage site is also disclosed. The method includes contacting an inactive channel-forming agent including a degenerate amino acid sequence substituted for the native activation sequence of the inactive channel-forming agent, with a protease, in the presence of red blood cells. Subsequently, plaque formation is detected, and the sequence the clone that generated the plaque obtained, using standard sequencing methods.

### BRIEF DESCRIPTION OF THE FIGURE

FIG. 1 is a line graph showing a decrease in absorbance, which corresponds to erythrocyte lysis in response to channel formation by active aerolysin. The solid line represents native proaerolysin activated with trypsin. The dashed line is the HIV variant of proaerolysin, activated with HIV protease. The dotted line is another aerolysin variant.

### SEQUENCE LISTING

The nucleic and amino acid sequences listed in the accompanying sequence listing are shown using standard letter abbreviations for nucleotide bases, and three-letter code for amino acids. Only one strand of each nucleic acid sequence is shown, but the complementary strand is understood as included by any reference to the displayed strand.

SEQ ID NOS: 1-8 show examples of amino acid HIV-1 protease cleavage sites.

SEQ ID NOS: 9 and 22 show examples of amino acid HCV NS3 protease cleavage sites.

SEQ ID NO: 10 shows an example of an amino acid HRV (Human Rhinoviruses) P2A protease cleavage site.

SEQ ID NOS: 11 and 12 show examples of HSV (Human herpes simplex virus) protease cleavage sites. SEQ ID NOS: 13 and 14 are a nucleotide sequence of an aerolysin gene from Aeromonas hydrophila and the corresponding amino acid sequence of the encoded protein, respectively.

SEQ ID NOS: 15 and 16 are nucleotide primers that can be used to amplify an aerolysin open reading frame (ORF).

SEQ ID NOS: 17 and 18 are nucleotide primers that can be used to introduce a HIV-1 protease recognition site into a channel-forming agent gene, such as a proaerolysin gene.

SEQ ID NOS 19 and 20 are nucleotide primers that can be used to introduce a HCV NS3 protease recognition site into a channel-forming agent gene, such as a proaerolysin gene.

SEQ ID NO: 21 is an amino acid sequence of a wild-type proaerolysin activation sequence from Aeromonas hydrophila.

### DETAILED DESCRIPTION OF SEVERAL EMBODIMENTS

### Abbreviations and Terms

The following explanations of terms and methods are provided to better describe the present disclosure and to guide those of ordinary skill in the art in the practice of the present disclosure. As used herein and in the appended claims, the singular forms “a” or “an” or “the” include plural references unless the context clearly dictates otherwise. For example, reference to “a liposome” includes a plurality of such liposomes and reference to “the protease” includes reference to one or more proteases and equivalents thereof known to those skilled in the art, and so forth.

Unless explained otherwise, all technical and scientific terms used herein have the same meaning as commonly understood to one of ordinary skill in the art to which this disclosure belongs.

Aerolysin: A bacterial toxin produced by members of the genus Aeromonas. However, the term aerolysin includes not only native toxins produced by Aeromonas members, but also to forms produced in other organisms by expressing a cloned Aeromonas aerolysin gene. In addition, aerolysin includes mutant, variant, fragment, fusion and polymorphic sequences, which retain the aerolysin biological ability, which is in one embodiment, the ability to from channels or pores in a lipid bilayer, leading to lysis of a liposome. This activity can be tested, for example, using a cytolytic or hemolysis assay. In one embodiment, aerolysin is a channel-forming cytolytic agent that can be activated by one or more specific proteases.

The precursor form of the aerolysin toxin is proaerolysin, and it is produced as a 52 kDa protein by the bacteria in the genus Aeromonas (Buckley, 1999). Proaerolysin is converted to aerolysin, the active form of the toxin, by proteolytic removal of a peptide approximately 40 amino acids long from its C-terminus. Aerolysin then oligomerizes into heptamers that can insert into cell membrane, forming 1.5 nm channels that lead to cell lysis. Structurally, proaerolysin is divided into two lobes (Parker et al., 1994). Proteolytic activation of proaerolysin occurs in a highly flexible loop at the carboxyl end of the large lobe (Howard et al., 1982). The flexible region contains an amino acid sequence that presents cleavage sites for a number of different proteases, including trypsin, chymotrypsin, furin, and protease K (Garland et al., 1988; Abrami et al., 1998). As a result, any of these proteases can activate the toxin, and once activated, aerolysin is resistant to further proteolysis.

The amino acid sequences of aerolysin produced by members of the Aeromonas genus are highly conserved. The nucleotide sequence of the aerolysin gene of Aeromonas hydrophila and the amino acid sequences of the encoded peptide as reported by Howard et al. (1987) are shown in SEQ ID NOS: 13 and 14, respectively. Nucleotide sequences of aerolysin genes from other members of the Aeromonas family and the corresponding amino acid sequences of the encoded proteins are known in the art (for example see Hirono et al., 1992; Hirono and Aoki, 1993; Husslein 1998; and Chopra et al., 1993).

In addition, mutant forms of aerolysin can be produced using standard mutagenesis techniques. Mutant forms of aerolysin include non-cytolytic forms, as those described in U.S. Pat. No. 5,798,218. Thus, in one embodiment, aerolysin includes all forms of the toxin which retain the ability to from channels in lipid bilayers, leading to cell lysis. Such mutant forms of aerolysin can be produced using site-directed or other standard mutagenesis-techniques, as described in Sambrook et al. (1989).

Because the nucleotide sequences of several aerolysin genes are known (see, for example, SEQ ID NO: 13), one skilled in the art will can produce the gene using the polymerase chain reaction (PCR) procedure, as described by Innis et al. (1990). Methods and conditions for PCR amplification of DNA are described in Innis et al. (1990) and Sambrook et al. (1989). The selection of PCR primers for amplification of the aerolysin gene can be made according to the portions of the gene which are desired to be amplified. Primers may be chosen to amplify small fragments of the gene or the entire gene molecule. Variations in amplification conditions may be required to accommodate primers of differing lengths; such considerations are well known in the art and are discussed in reference Innis et al. (1990). By way of example only, the entire aerolysin open reading frame may be amplified using the primers shown in SEQ ID NOS: 15 and 16. Template DNA for PCR amplification to produce the aerolysin gene can be extracted from Aeromonas cells using standard techniques (see Sambrook et al., 1989).

The cloned aerolysin gene can readily be ligated into bacterial expression vectors for production of the encoded aerolysin. Standard methods and plasmid vectors for producing prokaryotic proteins in bacteria are described in Sambrook et al. (1989). These methods facilitate large scale production of the protein and, if necessary, expression levels can be elevated by placing a strong, regulated promoter and an efficient ribosome binding site upstream of the cloned gene. Protease-deficient host cells are preferred since they yield higher levels of aerolysin.

The aerolysin gene may also be cloned into a suitable vector for mutagenesis. Mutations in the aerolysin gene may result in deletions or additions to the encoded amino acid sequence, or may be substitutions of one amino acid for another.

This disclosure includes mutant proaerolysins that have been modified to be selectively activated by a specific protease to be detected. Such modifications include, but are not limited to, (a) replacing native protease cleavage sites of a protoxin, such as proaerolysin, with a unique cleavage site recognizable only by a specific protease such as HIV-1 protease; (b) adding a peptide containing a unique cleavage site to an active channel-forming toxin such as alpha cytolysin of clostridium septicum which is inactivated as a result of the addition of the peptide and can only be activated by a specific protease that recognizing the cleavage site; (c) fusing two or more molecules of the same or different channel forming agents into an inactive form by a peptide linker bearing a unique cleavage site, and the fusion toxin can only be activated by a specific protease that recognizing the cleavage site.

This disclosure also includes mutant proaerolysin that has been modified to allow the determination of sequences that specific proteases recognize and cleave. Methods which can be used include generating multiple copies of proaerolysin with various unique protease cleavage sites, for example using the methods described above as well as by generating random protease cleavage sites through DNA or peptide synthesis, which would be used to replace the native protease cleavage site.

cDNA (complementary DNA): A piece of DNA lacking internal, non-coding segments (introns) and regulatory sequences which determine transcription. cDNA can be synthesized in the laboratory by reverse transcription from messenger RNA extracted from cells.

Channel-forming or Pore-forming Agent: An agent capable of forming transmembrane channels or pores in a lipid bilayer, such as the lipid bilayer of a liposome, which results lysis of the liposome. In one embodiment, a channel-forming agent is naturally-occurring. Naturally-occurring channel-forming agents are produced by many species of microorganisms, plants, and other organisms (Bayley, 1997). Examples of naturally-occurring channel-forming agents that can be used to practice the methods disclosed herein include, but are not limited to: aerolysin, alpha cytolysin of Staphylococcus aureas, alpha cytolysin of Clostridium septicum, Bacillus thuringenis toxin, colicin, complement, defensin, equinatoxin II, hemolysin, histolysin, listeriolysin, magainin, melittin, perfringolysin, perforin, pneumolysin, streptolysin O, and yeast killer toxin. Channel-forming agents also include mutants, variants, fragments, fusions and polymorphisms of any naturally-occurring toxin that retains cytolytic activity.

In an alternative embodiment, channel-forming agents include synthetic channel-forming agents. Examples include, but are not limited to: organic compounds such as valinomycin, Peterson's crown ethers, and other molecules, such as those described in Regen et al. (1989, herein incorporated by reference).

An inactive channel-forming agent is a channel-forming agent in an inactive form, which can be activated by a protease thus converting the inactive agent into an active lytic form. Some protein toxins, such as aerolysin, alpha cytolysin and Bacillus thuringenis toxins, exist in inactive forms, known as protoxins, in nature and can be activated by proteases. Inactive channel-forming agents also include mutants, variants, fragments, fusions and polymorphisms which retain the ability to be activated by a protease. This disclosure encompasses mutant inactive channel-forming agents, as well as a channel-forming agent comprising an attached compound, such as a nucleotide sequence, polypeptide, or antibody.

Biological activity can be determined by testing the compound's ability to form channels in natural or synthetic lipid bilayers, for example using a method which measures the release of intracellular contents from cells or liposomes pre-loaded with a dye, such as a fluorophore or chemiluminescent molecule, or radioactive label using for example a plate reader or spectrofluorimeter. In another embodiment, biological activity is determined by testing the compound's ability to form channels in an erythrocyte, for example using a method which measures the decrease in absorbance at 600 nm or 620 nm due to lysis of the erythrocytes.

The disclosure also includes analogues of naturally occurring channel-forming compounds. Differences between naturally-occurring compounds and their analogues can include amino acid sequence differences. There are various techniques to produce amino acid differences including site specific mutagenesis of nucleic acids using polymerase chain reaction (PCR) or other molecular biology techniques, and random mutagenesis by irradiation or exposure to mutagenic compounds. Other methods of producing analogues include in vivo or in vitro derivatization of polypeptides (acetylation or carboxylation), glycosylation modifications, and alterations in phosphorylation. Analogs can also have one or more peptide bonds replaced by a covalent bond that is not susceptible to peptidase cleavage.

Also included are modifications that allow the proform of the channel-forming agent to be activated by a cell associated substance or condition. These modifications can include the addition of a peptide containing an enzymatic or proteolytic cleavage site, chemically reactive groups, photoactivated groups, and metal binding sites.

The exemplary processes listed herein are not all-encompassing and the peptides of this invention can be produced by other processes.

Chemical synthesis: An artificial means by which one can make a protein or peptide. A synthetic protein or peptide is one made by such artificial means.

Comprises: A term that means “including.” For example, “comprising A or B” means including A or B, or both A and B, unless clearly indicated otherwise.

Cytolytic or Hemolytic Assay: Method used to measure liposome lysis, such as cell lysis. Cytolytic or hemolytic assay methods are well-known to those skilled in the art. In one embodiment, the assay is performed under physiological conditions relevant to a protease. Examples of cytolytic or hemolytic assays include, but are not limited to a hemolytic plaque assay or a hemolytic titer assay, assays which are well known to those skilled in the art.

Deletion: The removal of a sequence of a nucleic acid, for example DNA, the regions on either side being joined together.

DNA: Deoxyribonucleic acid. DNA is a long chain polymer which comprises the genetic material of most living organisms (some viruses have genes comprising ribonucleic acid, RNA). The repeating units in DNA polymers are four different nucleotides, each of which comprises one of the four bases, adenine, guanine, cytosine and thymine bound to a deoxyribose sugar to which a phosphate group is attached. Triplets of nucleotides, referred to as codons, in DNA molecules code for amino acid in a polypeptide. The term codon is also used for the corresponding (and complementary) sequences of three nucleotides in the mRNA into which the DNA sequence is transcribed.

DNA Construct: Any CDNA, genomic DNA, synthetic DNA or RNA. The term “construct” denotes a nucleic acid segment that may be single- or double-stranded, and that may be based on a complete or partial naturally occurring nucleotide sequence, such as a nucleic acid sequence encoding a channel-forming agent, for example an aerolysin gene. It is understood that such nucleotide sequences include intentionally manipulated nucleotide sequences, e.g., subjected to site-directed mutagenesis, and sequences that are degenerate as a result of the genetic code. All degenerate nucleotide sequences are included within the scope of the invention, so long sequence retains the functional activity of the non-degenerate sequence. For example, an aerolysin peptide encoded by a degenerate nucleotide sequence will maintain the ability to form channels or pores in lipid membranes.

Isolated: An isolated biological component (such as a nucleic acid, protein or organelle) is a component that had been substantially separated or purified away from other biological components in the cell of the organism in which the components naturally occur, for example, other chromosomal and extra-chromosomal DNA, RNA, proteins, and organelles. Nucleic acids and proteins that have been “isolated” include nucleic acids and proteins purified by standard purification methods. The term also embraces nucleic acids and proteins prepared by recombinant expression in a host cell, as well as chemically synthesized nucleic acids and proteins.

Liposome: A closed vesicle bounded by at least a single bilayer of phospholipids, wherein the space formed by the vesicle includes a solution. In one embodiment, a liposome has the properties described in U.S. Pat. No. 4,348,384 to Horikoshi et al. (herein incorporated by reference). In another embodiment, a liposome is a vesicle which is sensitive to at least one channel-forming agent. In one embodiment, liposomes are used to determine whether a protease is present in a sample.

In one embodiment, a liposome is a naturally-occurring vesicle, such as a cell. Examples of cells that can be used for the method disclosed herein include, but are not limited to cells from mammals, insects, plants, and microorganisms, such as those which can be cultured in vitro and are sensitive to channel-forming agents. Examples of cells include, but are not limited to, erythrocytes and T-lymphocytes. Lysis of erythorcytes can be detected by measuring the decrease in absorbance at 600 nm or 620 nm. Lysis of T-lymphocytes can be detected by measuring cell death.

In another embodiment, a liposome is a synthetic vesicle which comprises a lipid bilayer. In a particular embodiment, liposomes are modified to contain specific binding sites for a channel-forming agent such as glycosylphosphatidylinositol (GPI) anchored proteins. In another embodiment, a liposome includes a compound that can be detected upon release from the liposome, such as a radioactive marker, salt, nucleotide, polypeptide, or dye, such as a fluorophore. Standard techniques as demonstrated in Sambrook et al., (1989) can be used to make these manipulations.

Mammal: This term includes both human and non-human mammals. Similarly, the terms “patient,” “subject,” and “individual” includes both human and veterinary subjects. Examples of mammals include, but are not limited to: humans, pigs, cows, goats, cats, dogs, rabbits and mice.

Oligonucleotide: A linear polynucleotide sequence of up to about 200 nucleotide bases in length, for example a polynucleotide (such as DNA or RNA) which is at least about 6 nucleotides, for example at least 10, 15, 20, 50, 100 or 200 nucleotides long.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

ORF (open reading frame): A series of nucleotide triplets (codons) coding for amino acids without any termination codons. These sequences are usually translatable into a peptide.

Primers: Primers are short nucleic acids, such as DNA oligonucleotides about at least 15 nucleotides in length. Primers can be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, e.g., by PCR or other nucleic-acid amplification methods known in the art.

Methods for preparing and using primers are described, for example, in Sambrook et al. (1989); Ausubel et al. (1987); and Innis et al. (1990). PCR primer pairs can be derived from a known sequence, for example, by using computer programs intended for that purpose such as Primer (Version 0.5, 1991, Whitehead Institute for Biomedical Research, Cambridge, Mass.). One of skill in the art will appreciate that the specificity of a particular probe or primer increases with the length of the probe or primer. For example, a primer comprising 20 consecutive nucleotides will anneal to a target with a higher specificity than a corresponding primer of only 15 nucleotides. Thus, to obtain greater specificity, primers may be selected that comprise at least 10, 20, 25, 30, 35, 40, 50 or more consecutive nucleotides.

Polynucleotide: A linear nucleic acid sequence of any length. Therefore, a polynucleotide includes molecules which are at least 15, 50, 100, 200 or 400 (oligonucleotides) and also nucleotides as long as a full-length cDNA.

Promoter: An array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Protease: Proteases or proteinases are enzymes that cleave peptide bonds between amino acids. Particular proteases can function in digestion, blood clotting, fertilization, viral maturation, activation of zymogen, or formation and release of hormones and growth factors. In one embodiment, proteases can involved in a disease process, such as tissue injury, necrosis, inflammation, malignant tumors, rheumatoid arthritis, inflammatory bowel disease, gingival disease, glomerulonephritis, acute pancreatitis, Alzheimer's disease, cystic fibrosis, pulmonary emphysema, atherosclerosis, hypertension, and/or muscular dystrophy. Proteases can be found in mammalian cells, viruses, bacteria, fungi, insects and plants.

Some pathogenic viruses, bacteria, and/or fungi also rely on proteases for infection, replication, and/or maturation. For example, the aspartyl protease of HIV (also known as HIV-1 protease) that is translated as part of the Gag-Pol polypeptide, is responsible for its own processing and for releasing structural proteins and enzymes during viral replication and maturation in host cells. The serine protease NS3 of the hepatitis C virus (HCV) is responsible for the release of nonstructural proteins from the HCV polyproteins that are essential for the replication of the virus in the host cells. The viral-specific proteases of the herpes simplex virus (HSV) (EP Application No. 514,830; Liu and Roizman, 1991) and a related protease known as assemblin (Welch et al., 1991) of the cytomegalovirus (CMV) are known for their role in viral replication in the host cells.

Protease Cleavage Site: An amino acid sequence recognized by a protease that cleaves at that point. Unique cleavage sites include all the cleavage sites recognizable by broad range proteases such as trypsin-like or chymotrypsin-like enzymes or by specific or narrow range proteases. Some of the specific or narrow range proteases are often associated with certain processes of diseases or disorders such as tissue injury, necrosis, inflammation, repair, degeneration and infection.

In one embodiment, a cleavage site of interest is one that is specifically recognized by a protease associated with viral replication and maturation, bacterial and fungal infection, cystic fibrosis, blood clotting, hypertension, pulmonary, malignant tumors, rheumatoid arthritis, gingival disease, atherosclerosis, physical or chemical trauma, muscular dystrophy, and/or Alzheimer's disease. In a further embodiment, the protease cleavage sites include those that are specific to HIV-1 protease, HCV NS3 protease, HCV protease, HRV (Human Rhinoviruses) P2A protease, CMV protease, or HSV protease, and their active fragments or fusion proteins. The amino acid sequences of the cleavage sites of many proteases are known.

Examples of cleavage sites that are recognizable by proteases associated with some infectious diseases include, but are not limited to: HIV-1 protease cleavage sites shown in SEQ ID NOS: 1-8 (see Pichuantes et al., 1989), HCV NS3 protease cleavage sites shown in SEQ ID NOS: 9 and 22, an HSV (Human herpes simplex virus) protease cleavage sites shown in SEQ ID NOS: 11 and 12, and an HRV P2A protease cleavage site shown in SEQ ID NO: 10. HRVs are widespread, attack the upper respiratory tract in human and result in acute infections that lead to colds, coughs, sore throat, etc. and are generally referred to as colds.

Protease Inhibitors: A compound that decreases, such as inhibits, the activity of a specific protease or group of proteases. Inhibitors can be natural or synthetic. In one embodiment, protease inhibitors can be used to treat disorders that involve proteases, such as HIV, Squamous Cell Carcinomca, Alzheimer's disease and Hepatitis C.

Purified: The term purified does not require absolute purity; rather, it is intended as a relative term. Thus, for example, a purified channel-forming agent preparation is one in which the channel-forming agent is more pure than the channel-forming agent in its natural environment within a cell. For example, a preparation of a channel-forming agent is purified if the protein represents at least 50%, for example at least 70%, of the total protein content of the preparation. Methods for purification of proteins and nucleic acids are well known in the art. Examples of methods that can be used to purify a protein, such as a channel-forming agent, include, but are not limited to the methods disclosed in Sambrook et al. (Molecular Cloning: A Laboratory Manual, Cold Spring Harbor, N.Y. 1989, Ch. 17).

In one embodiment, aerolysin is in a purified form. Purified aerolysin is a preparation of aerolysin in which the aerolysin has been separated from substantially all of the cellular proteins (if produced by lysis of cells) or from substantially all proteins in the growth medium (if purified from growth medium following secretion of cells). In a particular embodiment, aerolysin will represent no less than 70% of the protein content of the preparation. However the aerolysin preparation may be constituted using a carrier protein, such as serum albumin, in which case aerolysin may represent less than 70% of the protein content of the preparation (Buckley, 1990).

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. A recombinant protein is one that results from expressing a recombinant nucleic acid encoding the protein.

RT: Room temperature

Sample: A material to be analyzed. In one embodiment, a sample is a biological sample. In one embodiment, a biological sample contains genomic DNA, cDNA, RNA, or protein obtained from the cells of a subject. Other examples of biological samples, include, but are not limited to: peripheral blood, serum, plasma, urine, cerebrospinal fluid, pleural fluid, synovial fluid, peritoneal fluid, gastric fluid, saliva, lymph fluid, interstitial fluid, sputum, stool, physiological secretions, tears mucus, sweat, milk, semen, seminal fluid, vaginal secretions, fluid from ulcers and other surface eruptions, blisters, and abscesses, tissue biopsy, surgical specimen, fine needle aspriates, amniocentesis samples, autopsy material, cell culture supernatant, fermentation supernatant, and tissue homogenates.

In another embodiment, a sample is an environmental sample, such as soil, water, or air. Environmental samples may be obtained from an industrial source, such as a building site, waste stream, water source, supply line, or production lot. Industrial sources also include fermentation media. In one embodiment the sample is concentrated prior to the assay.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least 70%, 75%, 80%, 85%, 90%, 95%, or even 99% sequence identity. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

Protein homologs are typically characterized by possession of at least 70%, such as at least 75%, 80%, 85%, 90%, 95% or even 98% sequence identity, counted over the full-length alignment with the amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG.

One of skill in the art will appreciate that these sequence identity ranges are provided for guidance only; it is possible that strongly significant homologs could be obtained that fall outside the ranges provided. Provided herein are the peptide homologs described above, as well as nucleic acid molecules that encode such homologs.

Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous peptides can, for example, possess at least 75%, 80%, 90%, 95%, 98%, or 9.9% sequence identity determined by this method. When less than the entire sequence is being compared for sequence identity, homologs can, for example, possess at least 75%, 85% 90%, 95%, 98% or 99% sequence identity over short windows of 10-20 amino acids. Methods for determining sequence identity over such short windows can be found at the NCBI web site. One of skill in the art will appreciate that these sequence identity ranges are provided for guidance only; it is possible that significant homologs or other variants can be obtained that fall outside the ranges provided.

Subject: Living multicellular vertebrate organisms, a category which includes, both human and veterinary subjects for example, mammals, rodents, and birds.

Variant, fragment, or fusion Sequences: The production of channel-forming agent can be accomplished in a variety of ways, using standard molecular biology methods. DNA sequences which encode for a protein or fusion protein, or a fragment or variant of a protein (for example a fragment or variant having 80%, 90% or 95% sequence identity to a channel-forming agent) can be engineered to allow the protein to be expressed in eukaryotic cells or organisms, bacteria, insects, and/or plants. To obtain expression, the DNA sequence can be altered and operably linked to other regulatory sequences. The final product, which contains the regulatory sequences and the channel-forming agent, is referred to as a vector. This vector can be introduced into eukaryotic, bacteria, insect, and/or plant cells. Once inside the cell the vector allows the protein to be produced.

A fusion channel-forming agent comprising a protein linked to other amino acid sequences can be generated. In one embodiment, the other amino acid sequences are no more than 10, 20, 30, or 50 amino acid residues in length.

One of ordinary skill in the art will appreciate that the DNA can be altered in numerous ways without affecting the biological activity of the encoded protein. For example, PCR can be used to produce variations in the DNA sequence which encodes an antigen. Such variants can be variants optimized for codon preference in a host cell used to express the protein, or other sequence changes that facilitate expression.

Vector: A nucleic acid molecule as introduced into a host cell, thereby producing a transformed host cell. A vector can include nucleic acid sequences that permit it to replicate in the host cell, such as an origin of replication. A vector can also include one or more selectable marker genes and other genetic elements known in the art.

Additional definitions of terms commonly used in molecular genetics can be found in Benjamin Lewin, Genes V published by Oxford University Press, 1994 (ISBN 0-19-854287-9); Kendrew et al (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Biochemical procedures described herein can be performed using standard laboratory methods as described in Molecular Cloning: A Laboratory Manual, 2^{nd} ed., vol. 1-3, ed. Sambrook et al., Cold Spring Harbour Laboratory Press, Cold Spring Harbor N.Y., 1989 (herein Sambrook et al., 1989); Current Protocols in Molecular Biology, ed. Ausubel et al., Greene Publishing and Wiley-Interscience, New York, 1992 (with periodic updates) (herein Ausubel et al., 1992) and Harlow and Lane, Antibodies: A Laboratory Manual, Cold Spring Harbor Laboratory, Cold Spring Harbor, N.Y., 1988 (herein Harlow and Lane, 1988), unless otherwise noted.

Herein disclosed is a method for detecting a protease in a sample. The method involves contacting an inactive channel-forming agent, which includes a protease cleavage site specific for the protease to be detected, with the sample and with a liposome, then measuring lysis of the liposome. The presence of liposome lysis indicates that protease is present in the sample, while absence of liposome lysis indicates the sample is free of detectable levels of the protease. In one embodiment, liposome lysis results from activation of the inactive channel-forming agent by proteolytic cleavage at the cleavage site by the protease in the sample.

In one embodiment of the disclosure, the method can be used to diagnosing diseases or disorders associated with a protease in a sample. For example, presence of a protease in the sample may indicate that the subject from whom the sample was obtained has a disease. Examples of these proteases include, but are not limited to, HIV-1 protease, HCV NS3 protease, human rhinovirus P2A protease, Herpes simplex virus (HSV) proteases, hepatitis proteases such as hepatitis A, C, E or G proteases, collagenase Type IV in human tumors, clostridiopeptidase A of the pathogenic bacterium C. histolyticum, and cathepsin D in the extracellular space in human malignant tissues. Other examples of proteases associated with specific viruses, include, but are not limited to feline immunodeficiency virus (FIV), feline calcivirus, yellow fever virus, bovine and ovine viral diarrhea virus, Japanese encephalitis virus and coronavirus infectious bronchitis virus.

In a particular embodiment, the disease is acquired immuno-deficiency syndrome (AIDS) and the protease detected is an HIV-1 protease. In another embodiment, the disease is hepatitis C and the protease detected is an HCV NS3 protease. In another embodiment, the disease is an upper respiratory tract infection and the protease is an HRV P2A protease. In yet another embodiment, the disease is herpes and the protease is an HSV protease. Examples of other diseases in which a protease cleavage site is recognized by a protease associated with the disease include, but are not limited to: Alzheimer's disease, cystic fibrosis, pulmonary emphysema, atherosclerosis, hypertension, and muscular dystrophy. Herein disclosed are modified channel-forming agents which include a peptide containing a protease cleavage site linked to a channel-forming agent. In one embodiment, the modified channel-forming agent is in an inactive form as a result of the addition of the peptide, but can be converted to an active form by a protease that cleaves at the cleavage site. In a particular embodiment, the inactive channel-forming agent is a naturally-occurring channel-forming toxin, such as a cytolytic toxin produced by bacteria, fungi, insects or plants. Examples of cytolytic toxins, include, but are not limited. to aerolysin, alpha cytolysin of Staphylococcus aureas, alpha cytolysin of Clostridium septicum, Bacillus thuringenis toxin, colicin, complement, defensin, equinatoxin II, hemolysin, histolysin, listeriolysin, magainin, melittin, perfringolysin, perforin, pneumolysin, streptolysin O and yeast killer toxin. In another embodiment, the naturally-occurring toxin is a naturally-occurring protoxin, such as proaerolysin, alpha cytolysin or Bacillus thuringenis toxin. In yet another particular embodiment, the inactive channel-forming agent is a synthetic channel-forming toxin, such as valinomycin or Peterson's crown ethers. Channel-forming agents can also include fragments, variants, fusions, or mutants of naturally occurring or synthetic toxins that retain cytolytic activity.

Also disclosed herein are inactive channel-forming agents wherein the native protease cleavage site of the inactive channel-forming agent is substituted for the protease cleavage site specific for the protease. In a particular embodiment, the inactive channel-forming agent is a proaerolysin including a protease cleavage site specific to an HIV-1 protease, such as a sequence comprising a sequence shown in SEQ ID NO: 1, 2, 3, 4 , 5, 6, 7, or 8, substituted for a native proaerolysin protease cleavage site. In yet another embodiment, the inactive channel-forming agent is a proaerolysin comprising a prbtease cleavage site specific to an HCV NS3 protease such as a sequence comprising a sequence shown in SEQ ID NO: 9 or 22, substituted for a native proaerolysin protease cleavage site. In a further embodiment, the inactive channel-forming agent is a proaerolysin comprising a protease cleavage site specific to an HRV P2A protease such as a sequence comprising a sequence shown in SEQ ID NO: 10, substituted for a native proaerolysin protease cleavage site. In yet another embodiment, the inactive channel-forming agent is a proaerolysin comprising a protease cleavage site specific to an HSV protease such as a sequence comprising a sequence shown in SEQ ID NO: 11 or 12, substituted for a native proaerolysin protease cleavage site.

In a particular embodiment, the inactive channel-forming agent is a modified channel-forming cytolytic toxin including a fusion of two or more cytolytic toxins, such as alpha cytolysin of clostridium septicum, colicin, complement, defensin, equinatoxin II, hemolysin, histolysin, listeriolysin, magainin, melittin, perfringolysin, perforin, pneumolysin, streptolysin O, or yeast killer toxin, and a linker peptide including a specific protease cleavage site.

Liposomes which can be used to practice the methods disclosed herein include, but are not limited to artificial or natural liposomes, such as a cell, for example mammalian, insect, fungal, or plant cells. In a particular embodiment, a liposome is a erythrocyte or T-lymphocyte. In one embodiment, the liposome sensitive to at least one channel-forming agent.

Samples that can be analyzed using the disclosed method include biological and/or environmental samples. Examples of biological samples, include, but are not limited to: peripheral blood, serum, plasma, urine, cerebrospinal fluid, pleural fluid, synovial fluid, peritoneal fluid, gastric fluid, saliva, lymph fluid, interstitial fluid, sputum, stool, physiological secretions, tears mucus, sweat, milk, semen, seminal fluid, vaginal secretions, fluid from ulcers and other surface eruptions such as a blister or abscess, tissue biopsy, surgical specimen, fine needle aspriates, amniocentesis samples, autopsy material, cell culture supernatant, fermentation supernatant, or tissue homogenate.

Lysis of a liposome can be measured using any standard assay. In one embodiment, lysis is measured using a cytolysis or hemolysis assay, such as a hemolytic plaque assay or a hemolytic titer assay.

In a particular embodiment, the present disclosure provides a sensitive assay which can be used to detect a protease in a sample, such as HIV-1 protease or HCV NS3 protease, wherein the concentration of protease is at very low concentrations in the samples. The method uses a modified proaerolysin and erythrocytes. Erythrocytes display specific receptors (glycosylphosphatidylinositol anchored proteins) to aerolysin, and as a result, are very sensitive to very low levels of the toxin (10⁻⁰ to 10⁻¹⁰ M). Naturally-occurring proaerolysin can be activated by a number of different proteases including trypsin, chymotrypsin, furin and protease K. The modified proaerolysin disclosed herein has had all of the native protease cleavage sites removed and replaced with a cleavage site specific to a protease, such as HIV-1 protease or HCV NS3 protease. Because of the high sensitivity of erythrocytes to aerolysin, this assay system can be used to detect, for example, 100 ng or less of HIV-1 protease or HCV NS3 protease.

Disclosed herein is a method of screening a test compound for a capacity to function as protease inhibitor. For example, the inhibitory effect of a test compound on a particular protease can be determined by contacting the test compound with the protease, a liposome, and an inactive channel-forming agent including a protease cleavage site specific for the protease thought to be inhibited by the test compound, followed by measuring lysis of the liposome. The inhibitory effect of the test compound is determined by comparing the lytic activity in the presence and absence of the test compound.

Also provided herein is a method for identifying a protease cleavage site. The method includes contacting an inactive channel-forming agent including a degenerate amino acid sequence substituted for the native activation sequence of the inactive channel-forming agent with a protease in the presence of red blood cells, detecting plaque formation; and obtaining the sequence of the clone that generated the plaque.

### EXAMPLE 1

### Preparation of Aerolysin and Proaerolysin

This example discloses methods for preparing proaerolysin and aerolysin in quantities sufficient for the methods described below, as described in Buckley and Howard (1988). Similar methods can be used to prepare any channel-forming agent of interest.

Proaerolysin can be isolated from cultural supernatants of Aeromonas salmonicida CB3 transformed with a proaerolysin gene (aerA) of Aeromonas hydrophila (see Genbank Accession No: M16495). A. salmonicida CB3 is a protease-deficient strain which can produce higher yields of proaerolysin. Although protease-deficient strains produce a higher yield of proaerolysin, other transformed strains of Aeromonas, as well as other host cells and non-transformed Aeromonas strains can also be used.

Culture supernatants are concentrated fifty-fold by ultra filtration and then centrifuged for two hours at 100,000×g to remove particulate matter. The supernatant is exchanged into 20 mM phosphate buffer containing 0.3 M NaCl, pH 6.0, by passing it over a Sephadex G25 column. The resulting mixture is applied to a hydroxyapatite column equilibrated in the same buffer. Proaerolysin is eluted from the column with a linear gradient formed by the starting buffer and 0.2 M phosphate containing 0.3 M NaCl, pH.6.0. Peak fractions are combined and the protein is precipitated by adding ammonium sulfate to 60% of saturation at 0° C. Following centrifugation, the precipitate is dissolved in 20 mM HEPES, pH 7.4, and applied to a Pharmacia DEAE Sepharose C16B column. Purified proaerolysin is eluted with a linear gradient formed by the starting buffer and 20 mM HEPES, 0.4 M NaCl.

If desired, aerolysin can be produced from the purified proaerolysin by adding trypsin to 1 μg/ml and incubating with end-over-end mixing for 15 minutes at room temperature (RT). Immobilized trypsin can be removed by brief centrifugation; soluble trypsin can be inhibited by adding soybean trypsin inhibitor to 10 μg/ml.

### EXAMPLE 2

### Preparation of Liposomes

This example describes methods that can be used to prepare artificial or synthetic liposomes. Such liposomes can be used to detect the presence or absence of a protease in a sample.

Non-cellular compounds sensitive to channel-forming cytolytic compounds can be used to determine the level of active toxin. For example, liposomes can be modified to be sensitive to channel-forming cytolytic compounds.

Liposomes, composed of phosphatidylcholine, or mixtures of phosphatidylcholine and other lipids, are prepared with incorporated placental alkaline phosphatase, which acts as a receptor for aerolysin. A dye such as carboxyfluorescein is entrapped in the liposomes during preparation. This can be accomplished by constructing liposomes by known methods (for example, see Nelson and Buckley, 2000). Briefly, lipid films containing 12 μmol of total lipid in the proportions 5 PD:3 PE:3 CH, 3 PC:3 CH:2 SM, 4 PC:3 PE:3 CH, and 3 PC:3 PE:3 CH:1 SM, were dried under nitrogen. Dried films were desiccated overnight and then rehydrated in 2 ml of 20 mM HEPES, 0.15 M NaCl, 100 mM carboxyfluorescein, pH 7.4. Liposomes containing sphingomyelin were rehydrated at 45° C., while the others were rehydrated at RT. Liposomes were rapidly frozen (−70° C. acetone bath) and thawed (45° C. water bath) six times. After freeze thawing, liposomes were passed through a 0.4 μm polycarbonate filter (Nucleopore) six times, using a Lipex Biomembrane extruder. The sphingomyelin liposomes were extruded at 45° C., and the others were extruded at RT.

GPI-anchored placental alkaline phosphatase (PLAP) was incorporated into the liposomes to act as a receptor for proaerolysin. PLAP first was purified using the following method. Human PLAP (25 mg) was dissolved in 50 ml of 1% Triton X-114 in PBS containing 1 mM phenylmethylsulfonyl fluoride by incubating for 20 minutes on ice. The extract was separated into detergent-rich and aqueous phases by warming the sample to 37° C. for 10 minutes and then centrifuging in a JA17 rotor (Beckman) for 10 minutes at 10,000 rpm and 23° C. The detergent-rich phase was cooled and diluted back to 1% triton X-114 by adding cold 20 mM HEPES, pH 7.4. Following warming and centrifuging to separate the phases once more, protein was precipitated from the detergent-rich phase by adding five volumes of acetone at −20° C. and incubating on ice for 30 minutes and then centrifuging at 5000 rpm for 30 minutes at 0° C. in a JA17 rotor. The acetone was decanted, and the pellet was dried for two hours under vacuum. The dried pellet was resuspended in 20 mM HEPES, pH 7.4, containing 1% octyl glucoside, and applied to a DEAE column equilibrated in the same buffer. The column was eluted with a salt gradient of 0-0.5 M NaCl in 20 mM HEPES, pH 7.4, 1% octyl glucoside. Enzyme activity, which was assayed using a standard alkaline phosphatase assay, according to the manufacturer's instructions (Sigma, St. Louis, Mo.), appeared at approximately 0.18 M salt. Silver staining after SDS-polyacrylamide gel electrophoresis produced a single band accounting for more than 95% of applied material. The purified protein had a specific activity of approximately 400 units/mg, slightly less than the specific activity reported previously for purified alkaline phosphatase lacking the GPI anchor (Chang et al., 1992).

Purified PLAP was incorporated into the liposomes using the following method. To determine optimum octyl glucoside concentrations for incorporation of GPI-anchored proteins into liposomes, the method of Nosjean and Roux (1999), was used. The turbidity of liposomes at 450 nm was monitored while increasing the concentration of octyl glucoside until the absorbance at 450 nm began to decrease. This detergent concentration (20 mM for 4 PC:3 PE:3 CH, 21 mM for 3 PC:3 PE:3 CH:l SM, 23 mM for 5 PC:3 PE:3 CH, and 30 mM for 3 PC:3 PE:3 CH:2 SM liposomes) was chosen for GPI-anchored protein incorporation. PLAP (9.5 μg) was incubated with 500 μl of 1.3 mM lipid. This mixture was dialyzed overnight against 20 mM HEPES, 0.15 M NaCl pH 7.4 at 4° C. (5 PC:3 PE:3 CH and 4 PC:3 PE:3 CH liposomes), or 22° C. (sphingomyelin-containing liposomes) to remove detergent and free carboxyfluorescein. Liposomes were then passed over a Sephacryl S-300 column (23 ml) in 20 mM HEPES, 0.15 M NaCl, to remove unincorporated PLAP and free dye. Phosphorous assays were performed on liposomes collected off of the column.

### EXAMPLE 3

### HIV-1 Protease Activated Proaerolysin

This example describes methods used to generate a proaerolysin channel-forming agent, which is sensitive to an HIV-1 protease. One skilled in the art will understand that other combinations of channel-forming agents and protease sensitive sequences (for example HCV NS3 as described in EXAMPLE 4) can be used to practice the methods disclosed herein. In addition, the sequence of the channel-forming agent can be a native sequence, as well as a variant, fragment, mutant, or fusion sequence, which retains the ability to form transmembrane channels or pores in a lipid bilayer.

A proaerolysin variant in which the normal activation sequence K⁴²⁷VRRAR (SEQ ID NO: 21) was replaced with the HIV-1 protease sensitive sequence QNYPIV (amino acids 2-7 of SEQ ID NO: 2) using recombinant PCR with oligonucleotide primers containing the desired codon changes. The primers shown in SEQ ID NOS: 17 and 18 were used to amplify and mutate the proaerolysin gene (see Genbank Accession No: M16495 for nucleotide and amino acid sequence of a wild-type proaerolysin from Aeromonas hydrophila ) from the cloned proaerolysin gene using standard PCR conditions as taught in Sambrook et al. (1989).

A 900 bp PCR fragment was generated. The final PCR product was digested using appropriate restriction enzymes and then ligated into the cloning vector pTZ18u for amplification according to standard procedures (See Sambrook et al., 1989). DNA sequencing was performed using standard methods to ensure the correct mutation was made. The insert was subsequently isolated from the cloning vector and subcloned into the broad-host-range plasmid pMMB66HE for expression in Aeromonas salmonicida. Recombinant clones were transferred into A. salmonicida strain CB3 by conjugation using the filter-mating technique, which is well known to those skilled in the art (for example see Figuski and Jelinski, 1979, herein incorporated by reference).

The HIV-1 sensitive proaerolysin variant was purified from culture supernatants of A. salmonicida containing the recombinant clone. After overnight (ON) induction, cells were removed by centrifugation and the supernatant concentrated by ultrafiltration. The concentrate was centrifuged to remove insoluble material, and desalted by column chromatography. This was followed by chromatography on hydroxyapatite, followed by chromatography on DEAE Sepharose, using the method described in Buckley (1990; herein incorporated by reference).

### EXAMPLE 4

### HCV NS3 Protease Activated Proaerolysin

This example describes methods used to generate a proaerolysin channel-forming agent, which is sensitive to an HCV NS3 protease. The methods used were similar to those described in EXAMPLE 3. One skilled in the art will understand that other channel-forming agents which are sensitive to an HCV NS3 protease using the methods disclosed herein.

A proaerolysin variant in which the normal activation sequence K⁴²⁷VRRAR (SEQ ID NO: 21) was replaced with the HCV NS3 protease sensitive sequence DEMRAC (SEQ ID NO: 22), using recombinant PCR with oligonucleotide primers containing the desired codon changes. The primers shown in SEQ ID NOS: 19 and 20 were used to amplify and mutate the proaerolysin gene from the cloned proaerolysin gene using standard PCR conditions as taught in Sambrook et al. (1989).

A 900 bp PCR fragment was generated. The final PCR product was digested using appropriate restriction enzymes and then ligated into the cloning vector pTZ18u for amplification according to standard procedures (See Sambrook et al., 1989). DNA sequencing was then carried out to ensure the correct mutation had been made. The insert was subsequently isolated from the cloning vector and subcloned into the broad-host-range plasmid pMMB66HE for expression in A. salmonicida. Recombinant clones were transferred into A. salmonicida strain CB3 by conjugation using the filter-mating technique as described above.

The HCV-sensitive proaerolysin variant was purified from culture supernatants of A. salmonicida containing the recombinant clone as described above in EXAMPLE 3.

### EXAMPLE 5

### Detection of HIV-1 Protease Using a Modified Proaerolysin

This example describes several different methods used to detect HIV-1 in a sample, using the proaerolysin variant generated in EXAMPLE 3. One skilled in the art will understand that any method which can detect lysis of a liposome, such as cell lysis, can be used to practice the methods disclosed herein. In addition, the methods disclosed in this example can be used to detect any protease of interest, using a channel-forming agent comprising a protease cleavage site specific for the protease to be detected.

Hemolytic Plate Assay

The HIV-1 proaerolysin variant generated in EXAMPLE 3 was incubated with varying concentrations of an HIV protease-containing sample in a microtiter plate (HIV-1 protease obtained from BACHEM, Torrance, Calif.). The HIV-1 proaerolysin variant (1.5×10⁻⁶ M) was diluted 1:16 in PBS to a final volume of 100 μl and added to the first column of wells of a 96 well plate. Various concentrations of HIV-protease containing samples, ranging from 10 to 500 ng of HIV-1 protease in a final volume of 100 μl, were added to each of the 8 wells of the first column of the microtiter plate, and the plate incubated for four hours.

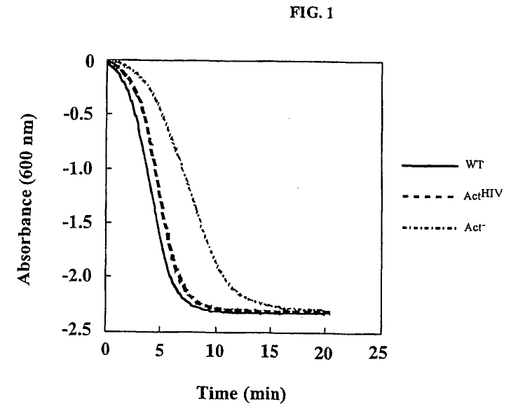

After incubation, the samples were serially diluted 1:2 by adding 100 μl of PBS to the first column of wells, mixing and transferring 100 μl sequentially to each of the wells in every row. Then, 100 μl of washed horse erythrocytes were added to all wells so that the final erythrocyte concentration was 0.4%. Absorbance at 620 nm was measured using a plate reader (Biotek Instruments, Inc., Winooski, Vt.) at 0, 5, 10, 15 and 20 minutes. Alternatively, absorbance at 600 nm can be measured using a spectrophotomer, as shown in FIG. 1. The decrease in absorbance shown in FIG.1 is due to lysis of the erythrocytes, which results when the proaerolysin variant is activated by the HIV-1 protease.

Tissue Culture Assay

The HIV-1 proaerolysin variant was serially diluted and incubated with the HIV-1 protease containing sample, as described above. Washed lymphocytes from the murine lymphocyte E14 cell line (R. Hyman, Salks Institute) were added to each well for a final volume of 10⁶ cells/ml. The EL4 cell line was grown in Dulbecco's modified Eagle's high glucose medium (DMEM) supplemented with bovine fetal clone I serum (10%, v/v), streptomycin (100 μml), and penicillin (100 units/ml) with 5% CO₂ at 37° C. Cells were harvested by centrifugation. The plate was incubated for one hour at 37° C. under 5% CO₂. Cell viability was measured by adding (3-(4,5-dimethylthiazol-2-yl)-5-(3-carboxymethoxyphenyl) -2-(4-sulfophenyl)-2H-tetrazolium and phenazine methosulfate, to final concentrations of 333 μg/ml and 7.66 μg/ml, respectively. The plate was then incubated at 37° C. with 5% CO₂ for four hours, after which the absorbance at 490 nm is measured over time using a plate reader (Biotek Instruments, Inc., Winooski, Vt.) as described above. If protease is present in the sample, absorbance at 490 nm decreases relative to a sample where there is no protease present, where the absorbance would not substantially change.

Liposome Release Assay

The HIV-1 proaerolysin variant is serially diluted and incubated with the HIV-1 protease containing sample, as described above.

Liposomes incorporating receptors for channel-forming compounds in their membrane and entrapping a reporter molecule, such as a dye, such as carboxyfluorescein, are prepared according to standard procedures as detailed in Sambrook et al., 1989, and as described above in EXAMPLE 2.

The modified liposomes are added to a four ml, 1 cm cuvette. Carboxyfluorescein release can be measured using a Photon technology spectrofluorimeter at 37° C. at 0, 5, 10, 15 and 20 minutes, using an excitation wavelength of 490 nm, and an emission wavelength of 520 nm. An increase in absorbance at 520 nm indicates the presence of a protease, due to release of carboxyfluorescein from the liposome. Little or no change in the absorbance at 520 nm indicates that protease is not present at detectable levels.

### EXAMPLE 6

### Detection of HCV NS3 Protease Using a Modified Proaerolysin

This example describes several different methods which can be used to detect HCV in a sample, using the proaerolysin variant generated in EXAMPLE 4. The methods are essentially those described in EXAMPLE 5, except that a different proaerolysin variant is used.

Hemolytic Plate Assay

The HCV NS3 proaerolysin variant is incubated with varying concentrations of a sample suspected of containing an HCV NS3 protease, in a microtiter plate. The HCV NS3 proaerolysin variant (1.5×10⁻⁶ M) is diluted 1:16 in PBS to a final volume of 100 μl and added to the first column of wells of a 96 well plate. Appropriate concentrations of a sample thought to contain an HCV NS3 protease in a final volume of 100 μl are added to each of the eight wells of the first column of the microtiter plate, and the plates incubated for four hours.

Following incubation, the samples are serially diluted, washed erythrocytes added, and absorbance at 620 nm or 600 nm measured as described above in EXAMPLE 5.

Tissue Culture Assay

An HCV NS3 proaerolysin variant is serially diluted and incubated with a test sample which may contain an HCV NS3 protease as described above. Murine lymphocytes from the E14 cell line are added, and cell viability measured by adding (3-(4,5-dimethylthiazol-2-yl)-5-(3-carboxymethoxyphenyl)-2-(4-sulfophenyl)-2H-tetrazolium and phenazine methosulfate as described above. The plate is then incubated at 37° C. with 5% CO₂ for four hours, after which the absorbance at 490 nm is measured as described above.

Liposome Release Assay

An HCV NS3 proaerolysin variant is serially diluted and incubated with a test sample which may contain an HCV NS3 protease as described above. Liposomes incorporating receptors for pore-forming compounds in their membrane and entrapping a dye such as carboxyfluorescein are added to a cuvette, and carboxyfluorescein release measured using the methods described above.

### EXAMPLE 7

### Screening Method

This example describes several different methods which can be used to determine whether a compound functions as a protease inhibitor, such as an HIV-1 protease inhibitor or an HCV protease inhibitor. One skilled in the art will understand that any method which can detect lysis of a liposome, such as cell lysis, can be used to practice the methods disclosed herein. In addition, the methods disclosed in this example can be used to detect any protease inhibitor of interest, using a channel-forming agent comprising a protease cleavage site specific for the protease inhibited by the potential protease inhibitor to be tested.

Hemolytic Plate Assay

Protease is added to each well of a 96 well plate. The concentration of protease used will depend on the activity of the protease. Enough protease is added to allow activation of a pro-form of a channel forming agent. Serial dilutions of known and/or potential protease inhibitors are added to the wells for a final volume of 100 μl. The plate is incubated for about four hours at 37° C. A channel-forming agent variant comprising a protease cleavage site specific for the protease inhibited by the known and/or potential protease inhibitor, such as the HIV-1 proaerolysin variant described in EXAMPLE 3 or the HCV proaerolysin variant described in EXAMPLE 4, is added to each of the wells for a final concentration of 10⁻⁶ M and incubated at 37° C. for four hours. A parallel control containing all the components except for the protease inhibitor is also prepared. This enables the results to be compared between the presence and absence of the prospective inhibitor.

After incubation, 100 μl of washed horse erythrocytes are added to all wells so that the final erythrocyte concentration is 0.4%. Absorbance at 600 or 620 nm is measured at 0, 5, 10, 15 and 20 minutes as described above.

Tissue Culture Assay

A channel-forming variant is serially diluted and incubated with a protease and a known and/or potential protease inhibitors as noted above. Murine lymphocytes from the E14 cell line are added, and cell viability measured by adding (3-(4,5-dimethylthiazol-2-yl)-5-(3-carboxymethoxyphenyl)-2-(4-sulfophenyl)-2H-tetrazolium and phenazine methosulfate as described in EXAMPLE 5. The plate is then incubated at 37° C. with 5% CO₂ for about four hours, after which the absorbance at 490 nm is measured as described in EXAMPLE 5.

Liposome Release Assay

A channel-forming variant is serially diluted and incubated with a protease and and a known and/or potential protease inhibitors as noted above. Liposomes incorporating receptors for pore-forming compounds in their membrane and entrapping a dye such as carboxyfluorescein are added to a cuvette, and carboxyfluorescein release measured using the methods described above in EXAMPLE 5.

### EXAMPLE 8

### Identifying Protease Cleavage Sites

This example describes methods that can be used to identify a sequence recognized and cleaved by a protease.

A DNA construct comprising an inactive channel-forming agent sequence, such as a proaerolysin sequence, and a degenerate protease cleavage site, can be generated as follows. Proaerolysin variants in which the normal activation sequence K⁴²⁷VRRAR (SEQ ID NO: 21) is replaced with a degenerate amino acid sequence using standard recombinant PCR methods, for example those described in Sambrook et al. (1989). Briefly, a completely degenerate 18 bp oligonucleotide is synthesized using standard methods, to replace the nucleotide sequence encoding the normal activation sequence. One skilled in the art will understand that longer oligonucleotide sequences comprising the 18 bp oligonucleotide can also be used. Restriction enzymes are used to remove the normal activation sequence from a proaerolysin gene contained in the cloning vector pTZ18u and the degenerate oligonucleotide ligated into the vector. The aerolysin insert is isolated from the cloning vector and subcloned into the broad-host-range plasmid pMMB66HE for expression in A. salmonicida. Recombinant clones are transferred into A. salmonicida strain CB3 by conjugation using the filter-mating technique as described above in EXAMPLE 3.

The recombinant A. salmonicida clones are grown on plates containing red blood cells. The desired protease is distributed onto the plates at varying concentrations in a total volume of 100 μl. The plate is incubated at 37° C. for four days to allow for the appearance of hemolytic plaques. The clones which resulted in plaque formation are isolated and the protease recognition site is sequenced, using standard sequencing methods.

In view of the many possible embodiments to which the principles of my disclosure may be applied, it should be recognized that the illustrated embodiments are only particular examples of the disclosure and should not be taken as a limitation on the scope of the disclosure. Rather, the scope of the disclosure is in accord with the following claims. I therefore claim as my invention all that comes within the scope and spirit of these claims.

### REFERENCES

Abrami et al., J. Biol. Chem. 49:32656-61, 1998.
Ausubel et al. (ed.), Current Protocols in Molecular Biology, Greene Publishing and Wiley-Interscience, New York (with periodic updates), 1987
Bayley, Sci. Am. 277:340-344, 1997.
Buckley, The comprehensive sourcebook of bacterial toxins. 362- 372, 1999.
Buckley, Biochem. Cell Biol. 68:221-4, 1990.
Buckley and Howard, Methods Enzymol. 165:193-9, 1988.
Chang et al., Eur. J. Biochem. 209:241-7, 1992.
Chopra et al., Can. J. Microbiol. 39:513-23, 1993.
Dasmahapatra et al., Proc. Natl. Acad. Sci. USA, 89:4159-62, 1992.
Figuski and Jelinski, Proc. Natl. Acad. Scl. USA, 76:1648-52, 1979.
Garland et al., Infect. Immun. 56:1249-53, 1988.
Hirono et al., Microb. Pathog. 13:433-46, 1992.
Hirono and Aoki, Microb. Pathog. 15:269-82, 1993.
Hirowatari et al., Anal. Biochem., 225:113-20, 1995.
Howard et al., Biochern. 21:1662-7, 1982.
Howard et al., J. Bacteriol. 169:2869-2871, 1987.
Husslein et al. (1998), Mol. Microbiol. 2:507-517.
Innis et al., PCR Protocols: A Guide to Methods and Applications, Academic Press: San Diego, 1990.
Liu and Roizman., J. Virol., 65:5149, 1991.
Murray et al., Gene, 134:123-8, 1993.
Nelson and Buckley, J. Biol. Chem. 275:19839-43, 2000.
Nosjean and Roux. Eur. J. Biochem. 263:865-870, 1999.
Parker et al., Nature 367:292-295, 1994.
Pichuantes et al. 1989. “Recombinant HIV-1 protease secreted by Sacchromyces cervisiae correctly processes myristylated gag polyprotein” in “Proteins: Structure, Function, and Genetics 6, 324-337.
Regen et al., Biochem. Biophys. Res. Commun. 159:566-571, 1989.
Sambrook et al. (ed.), Molecular Cloning: A Laboratory Manual, 2nd ed., vol. 1-3, Cold Spring Harbor Laboratory Press, Cold Spring Harbor, N.Y., 1989;
Smith et al., Proc. Natl. Acad. Sci. USA, 88:5159-62, 1991.
Welch et al., Proc. Natl. Acad. Sci. USA, 88:10792, 1991.

1. A method for detecting a protease in a sample comprising:
contacting an inactive channel-forming agent comprising a protease cleavage site specific for the protease, with the sample, and with a liposome; and
measuring lysis of the liposome.

2. The method of claim 1, wherein the presence of liposome lysis indicates the presence of the protease in the sample, and wherein the absence of liposome lysis indicates the absence of the protease in the sample.

3. The method of claim 1, wherein the liposome lysis results from activation of the inactive channel-forming agent by proteolytic cleavage at the cleavage site by the protease in the sample.

4. The method of claim 1, wherein the presence of the protease is indicative of the presence of a disease in a subject from whom the sample was obtained.

5. The method of claim 1, wherein the protease is an human immuno-deficiency virus-1 (HIV-1) protease.

6. The method of claim 4, wherein the disease is acquired immuno-deficiency syndrome (AIDS) and the protease is an HIV-1 protease.

7. The method of claim 5, wherein the inactive channel-forming agent is a proaerolysin comprising a protease cleavage site specific to an HIV-1 protease substituted for a native proaerolysin protease cleavage site.

8. The method of claim 7, wherein the protease cleavage site specific to the HIV-1 protease comprises a sequence shown in SEQ ID NO: 1, 2, 3, 4 , 5, 6, 7, or 8.

9. The method of claim 1, wherein the protease is an HCV NS3 protease.

10. The method of claim 4, wherein the disease is hepatitis C and the protease is an HCV NS3 protease.

11. The method of claim 9, wherein the inactive channel-forming agent is a proaerolysin comprising a protease cleavage site specific to an HCV NS3 protease substituted for a native proaerolysin protease cleavage site.

12. The method of claim 11, wherein the protease cleavage site specific to the HCV NS3 protease comprises a sequence shown in SEQ ID NO: 9 or 22.

13. The method of claim 1, wherein the protease is a human rhinovirus (HRV) P2A protease.

14. The method of claim 4, wherein the disease is an upper respiratory tract infection and the protease is an HRV P2A protease.

15. The method of claim 14, wherein the inactive channel-forming agent is a proaerolysin comprising a protease cleavage site specific to an HRV P2A protease substituted for a native proaerolysin protease cleavage site.

16. The method of claim 11, wherein the protease cleavage site specific to the HRV P2A protease comprises a sequence shown in SEQ ID NO: 10.

17. The method of claim 1, wherein the protease is a herpes simplex virus (HSV) protease.

18. The method of claim 4, wherein the disease is herpes and the protease is an HSV protease.

19. The method of claim 18, wherein the inactive channel-forming agent is a proaerolysin comprising a protease cleavage site specific to an HSV protease substituted for a native proaerolysin protease cleavage site.

20. The method of claim 19, wherein the protease cleavage site specific to the HSV protease comprises a sequence shown in SEQ ID NO: 11 or 12.

21. The method of claim 1, wherein the inactive channel-forming agent is a naturally-occurring toxin.

22. The method of claim 22, wherein the inactive channel-forming agent is a cytolytic toxin produced by bacteria, fungi, insects or plants.

23. The method of claim 22, wherein the naturally-occuring toxin is aerolysin, alpha cytolysin of Staphylococcus aureas, alpha cytolysin of Clostridium septicum, Bacillus thuringenis toxin, colicin, complement, defensin, equinatoxin II, hemolysin, histolysin, listeriolysin, magainin, melittin, perfringolysin, perforin, pneumolysin, streptolysin O or yeast killer toxin.

24. The method of claim 22, wherein the naturally-occurring toxin is a naturally-occurring protoxin.

25. The method of claim 24, wherein the naturally-occurring protoxin is a proaerolysin, alpha cytolysin or Bacillus thuringenis toxin.

26. The method of claim 25, wherein the naturally-occurring protoxin is proaerolysin.

27. The method of claim 1, wherein the inactive channel-forming agent is a synthetic toxin.

28. The method of claim 27, wherein the synthetic toxin is valinomycin or Peterson's crown ethers.

29. The method of claim 1, wherein a native protease cleavage site of the inactive channel-forming agent is substituted for the protease cleavage site specific for the protease.

30. The method of claim 29, wherein the inactive channel-forming agent is a modified channel-forming cytolytic toxin comprising a fusion of two or more cytolytic toxins and a linker peptide comprising a specific protease cleavage site.

31. The method of claim 30, wherein the cytolyic toxin is an alpha cytolysin of clostridium septicum, colicin, complement, defensin, equinatoxin II, hemolysin, histolysin, listeriolysin, magainin, melittin, perfringolysin, perforin, pneumolysin, streptolysin O, or yeast killer toxin.

32. The method according to claim 1, wherein the protease cleavage site is recognized by a protease associated with Alzheimer's disease, cystic fibrosis, pulmonary emphysema, atherosclerosis, hypertension, or muscular dystrophy.

33. The method of claim 1, wherein the liposome is an artificial liposome.

34. The method of claim 1, wherein the liposome is a cell.

35. The method of claim 34, wherein the cell is a mammalian cell.

36. The method of claim 34, wherein the cell is a erythrocyte or T-lymphocyte.

37. The method of claim 34, wherein the cell is an insect, fungal, or plant cell.

38. The method of claim 1 wherein lysis is measured using a cytolysis or hemolysis assay.

39. The method of claim 38, wherein lysis is measured using a hemolytic plaque assay.

40. The method of claim 38, wherein lysis is measured using a hemolytic titer assay.

41. The method of claim 1, wherein the sample is a biological or environmental sample.

42. The method of claim 41, where the biological sample is peripheral blood, serum, plasma, urine, cerebrospinal fluid, pleural fluid, synovial fluid, peritoneal fluid, gastric fluid, saliva, lymph fluid, interstitial fluid, sputum, stool, physiological secretions, tears mucus, sweat, milk, semen, seminal fluid, vaginal secretions, fluid from ulcers and other surface eruptions such as a blister or abscess, tissue biopsy, surgical specimen, fine needle aspriates, amniocentesis samples, autopsy material, cell culture supernatant, fermentation supernatant, or tissue homogenate.

43. A method of detecting an HIV-1 protease in a sample comprising:
contacting an inactive channel-forming agent comprising an HIV-1-specific protease cleavage site specific, a sample, and a liposome; and
detecting the presence of the HIV-1 protease by measuring lysis of the liposome caused by activation of the inactive channel-forming agent by the HIV-1 protease.

44. A method for screening a test compound for a capacity to function as protease inhibitor comprising:
contacting an inactive channel-forming agent comprising a protease cleavage site specific for a protease inhibited by the protease inhibitor, with the test compound, protease, and a liposome;
measuring lysis of the liposome; and
comparing liposome lysis to a sample containing no test compound.

45. A method for identification of a protease cleavage site comprising:
contacting an inactive channel-forming agent comprising a degenerate amino acid sequence substituted for the native activation sequence of the inactive channel-forming agent with a protease in the presence of red blood cells;
detecting plaque formation; and
obtaining a sequence of a clone that generated a plaque.


SEQUENCE LISTING (22 sequences)

SEQ ID NO: 1 - protein, 10 aa, Artificial Sequence
  HIV-1 protease cleavage site
VSFNFPQITL

SEQ ID NO: 2 - protein, 8 aa, Artificial Sequence
  HIV-1 protease cleavage site
SQNYPIVQ

SEQ ID NO: 3 - protein, 8 aa, Artificial Sequence
  HIV-1 protease cleavage site
ARVLAEAM

SEQ ID NO: 4 - protein, 8 aa, Artificial Sequence
  HIV-1 protease cleavage site
ANIMMQRG

SEQ ID NO: 5 - protein, 8 aa, Artificial Sequence
  HIV-1 protease cleavage site
PGNFLQSR

SEQ ID NO: 6 - protein, 8 aa, Artificial Sequence
  HIV-1 protease cleavage site
SFNFPQIT

SEQ ID NO: 7 - protein, 8 aa, Artificial Sequence
  HIV-1 protease cleavage site
QITLWQRP

SEQ ID NO: 8 - protein, 8 aa, Artificial Sequence
  HIV-1 protease cleavage site
RKVLFLNG

SEQ ID NO: 9 - protein, 10 aa, Artificial Sequence
  HCV NS3 protease cleavage site
GAATEDVVCC

SEQ ID NO: 10 - protein, 16 aa, Artificial Sequence
  Human Rhinoviruses P2A protease cleavage site
TRPIITTAGPSDMYVH

SEQ ID NO: 11 - protein, 8 aa, Artificial Sequence
  Human herpes simplex virus protease cleavage site
VVNASARL

SEQ ID NO: 12 - protein, 10 aa, Artificial Sequence
  Human herpes simplex virus protease cleavage site
GVVNASARLA

SEQ ID NO: 13 - DNA, 2346 bp, Aeromonas hydrophila
  CDS (532)..(1989)
cgccccgagtcagctgcggccgttcactcgcgacgggcacaggccccttgcttgcggtggccggtcactcgctgcaattgcaggggttgggcacaatcaccttcgatgccggcacccgctggctcaacggcggtcccgccgatctgcaaccgggtcgccaactggtgctgagccgcgatgaaacgggtcgggcaaccgagatcctgatccccaaccccgaggatgaaccggaataaggatcatgcagccaaacgcttaatatttattttgctaaattagaaatttcttttttatctatattccaaaagatgattaagtgacgaataaaataatagagcgagtgctctgatattatatcaatcaatattgaatgaagttcaatttatgattttgttaatatattgcgcatattaaaatgtgggctggatcgcatattgagattaatctcactgatattgtcgtactcacatgccacccgctgatatataaggttggtgaatgcatgtcaatgttcaatatattggggttgctatgcaaaaaataaaactaactggcttgtcattaatcatatccggcctgctgatggcacaggcgcaagcggcagagcccgtctatccagaccagcttcgcttgttttcattgggccaaggggtctgtggcgacaagtatcgccccgtcaatcgagaagaagcccaaagcgttaaaagcaatattgtcggcatgatggggcaatggcaaataagcgggctggccaacggctgggtcattatggggccgggttataacggtgaaataaaaccagggacagcgtccaatacctggtgttatccgaccaatcctgttaccggtgaaataccgacactgtctgccctggatattccagatggtgacgaagtcgatgtgcagtggcgactggtacatgacagtgcgaatttcatcaaaccaaccagctatctggcccattacctcggttatgcctgggtgggcggcaatcacagccaatatgtcggcgaagacatggatgtgacccgtgatggcgacggctgggtgatccgtggcaacaatgacggcggctgtgacggctatcgctgtggtgacaagacggccatcaaggtcagcaacttcgcctataacctggatcccgacagcttcaagcatggcgatgtcacccagtccgaccgccagctggtcaagactgtggtgggctgggcggtcaacgacagcgacaccccccaatccggctatgacgtcaccctgcgctacgacacagccaccaactggtccaagaccaacacctatggcctgagcgagaaggtgaccaccaagaacaagttcaagtggccactggtgggggaaacccaactctccatcgagattgctgccaatcagtcctgggcgtcccagaacgggggctcgaccaccacctccctgtctcagtccgtgcgaccgactgtgccggcccgctccaagatcccggtgaagatagagctctacaaggccgacatctcctatccctatgagttcaaggccgatgtcagctatgacctgaccctgagcggcttcctgcgctggggcggcaacgcctggtatacccacccggacaaccgtccgaactggaaccacaccttcgtcataggtccgtacaaggacaaggcgagcagcattcggtaccagtgggacaagcgttacatcccgggtgaagtgaagtggtgggactggaactggaccatacagcagaacggtctgtctaccatgcagaacaacctggccagagtgctgcgcccggtgcgggcggggatcaccggtgatttcagtgccgagagccagtttgccggcaacatagagatcggtgctcccgtgccgctcgcggctgacagcaaggtgcgtcgtgctcgcagtgtggacggcgctggtcaaggcctgaggctggagatcccgctcgatcgcgaagagctctccgggcttggcttcaacaagtcagcctcagcgtgacccctgctgccaatcaataacggcagcgcgttgtagtgatggaaccgggcctctgtggcccggtttttgtttgcactggtcgggcttgttaaaggcttgtgctttccatttccccacttatactggcgccatcttgtcggagtgccaaccgtcgaacgacgcgaggctgagaccgttaattcgggatccgtgcaacctcatcaggctagcacctgcgaagggaaacaagggtaacttgcgggttgccgcgccgggggagggacaagcctctccgcgtcatcaagaggagccattcctcgatgagtcagggcgcacaagagggactctgtcccgtccggtctgcccaggaggggc

SEQ ID NO: 14 - protein, 486 aa, Aeromonas hydrophila
MQKIKLTGLSLIISGLLMAQAQAAEPVYPDQLRLFSLGQGVCGDKYRPVNREEAQSVKSNIVGMMGQWQISGLANGWVIMGPGYNGEIKPGTASNTWCYPTNPVTGEIPTLSALDIPDGDEVDVQWRLVHDSANFIKPTSYLAHYLGYAWVGGNHSQYVGEDMDVTRDGDGWVIRGNNDGGCDGYRCGDKTAIKVSNFAYNLDPDSFKHGDVTQSDRQLVKTVVGWAVNDSDTPQSGYDVTLRYDTATNWSKTNTYGLSEKVTTKNKFKWPLVGETQLSIEIAANQSWASQNGGSTTTSLSQSVRPTVPARSKIPVKIELYKADISYPYEFKADVSYDLTLSGFLRWGGNAWYTHPDNRPNWNHTFVIGPYKDKASSIRYQWDKRYIPGEVKWWDWNWTIQQNGLSTMQNNLARVLRPVRAGITGDFSAESQFAGNIEIGAPVPLAADSKVRRARSVDGAGQGLRLEIPLDREELSGLGFNKSASA

SEQ ID NO: 15 - DNA, 27 bp, Artificial Sequence
  Primer
atgcaaaaaataaaactaactggcttg

SEQ ID NO: 16 - DNA, 27 bp, Artificial Sequence
  Primer
cgctgaggctgacttgaacggaagccc

SEQ ID NO: 17 - DNA, 34 bp, Artificial Sequence
  Primer
cagaactatccgatcgtgagtgtggacggcgctg

SEQ ID NO: 18 - DNA, 34 bp, Artificial Sequence
  Primer
cacgatcggatagttctggctgtcagccgcgagc

SEQ ID NO: 19 - DNA, 42 bp, Artificial Sequence
  Primer
ggctgacagtgatgagatgcgtgcttgtagtgtggacggcgc

SEQ ID NO: 20 - DNA, 42 bp, Artificial Sequence
  Primer.
gcgccgtccacactacaagcacgcatctcatcactgtcagcc

SEQ ID NO: 21 - protein, 6 aa, Aeromonas hydrophila
KVRRAR

SEQ ID NO: 22 - protein, 6 aa, Artificial Sequence
  HCV NS3 protease cleavage site.
DEMRAC